(19) United States
(12) Patent Application Publication
(10) Pub. No.: US 2007/0087432 A1
(43) Pub. Date: Apr. 19, 2007

(54) Smallpox vaccine series 77

(76) Inventor: John Ames Harrington, Hollywood, FL (US)

Correspondence Address: JOHN AMES HARRINGTON, 1839 WASHINGTON ST., HOLLYWOOD, FL, 33020-6043, US

(21) Appl. No.: 11/409,598

(22) Filed: Apr. 24, 2006

Related U.S. Application Data
(63) Continuation-in-part of application No. 10/446,200, filed on Sep. 5, 2003, now abandoned.

(30) Foreign Application Priority Data
Sep. 30, 2004 (CA) 2481,716

(51) Int. Cl. C12P 21/06; C12N 15/00

(52) U.S. Cl. 435/320.1; 435/69.1

(57) ABSTRACT

Herpes is treated by administering a smallpox vaccination and by repeatedly administering the vaccination after waiting a scab, formed after a blister at the vaccination site, falls off, with the treatment being completed when the blister and scab fail to form.

### TECHNICAL DISCLOSURE

### Using Wyeth Undiluted Smallpox Vaccine

In accordance with the invention, for treatment of a patient having the herpes virus, type 1 or type 2, a normal smallpox vaccination is administered, causing a blister to form and to be followed by the formation of a scab at the vaccination site. After the scab falls off, but certainly within 24-48 hours, a normal smallpox vaccination is administered again. This process is repeated, with the blister and scab becoming smaller each time, until there is no new visible blister and scab following the vaccination, indicating that the process is complete.

This treatment induces immunity within the patient to the herpes virus, a form of poxviridae virus, alleviating the effects of the associated disease. However, the virus is contagious until the process is completed. The process takes 4-6 weeks to complete, or longer in severe cases. The patient should not touch the blister or scab at the vaccination site. Scarring may be reduced by applying a vaccination to a site of a former childhood vaccination.

For example, a patient who was diagnosed with severe genital herpes in 1974 was treated with repeated small pox vaccinations until a visible blister was no longer produced at the vaccination site. Herpes sores disappeared in 1974 and have no longer reappeared since then.

In accordance with the invention, for treatment of a patient having the herpes virus, type 1 or type 2, a normal smallpox vaccination is administered, causing a blister to form and to be followed by the formation of a scab at the vaccination site. After the scab falls off, but certainly within 24-48 hours, a normal smallpox vaccination is administered again. This process is repeated, with the blister and scab becoming smaller each time, until there is no visible blister and scab following the vaccination indicating that the process is complete.

This treatment includes immunity within the patient to the herpes virus, a form of poxviridae virus, alleviating the effects of the associated disease. However, the virus is contagious until the process is completed. The process takes 4-6 weeks to complete, or longer in severe cases. The patient should not touch the blister or scab at vaccination site. Scarring may be reduced by applying a vaccination to a site of a former childhood vaccination.

For example, a patient who was diagnosed with severe genital herpes in 1974 was treated with repeated smallpox vaccination in series as explained above until a visible blister was no longer produced at the vaccination site. Herpes sores disappeared in 1974 and have not reappeared since then.

1. A method: a new use for an old product using Wyeth smallpox vaccine in series as explained in the technical disclosure

2. The method for treatment of a patient having a disease caused by a poxviridae virus, wherein the treatment comprises:
a. Administering a smallpox vaccination to the patient at a vaccination site;
b. Determining whether a blister and scab form the vaccination site;
c. In responding to determining that a blister and scab form at the vaccination site, repeating steps a) through c) after the scab falls off.

3. The method claim, wherein step a) is repeated within twenty-four to forty-eight hours after the scab falls off

4. The method claim, wherein the vaccination site is a a site previously used for a childhood vaccination.

5. A method for treatment of a patient having herpes, wherein the treatment comprises:
a. Administering a smallpox vaccination to the patient at a vaccination site;
b. Determining whether a blister and scab form at the vaccination site;
c. In response to determining that a blister and scab form at the vaccination site, repeating steps a) through c) after the scab falls off.

6. A method: Anecdotal evidence points to poxviridae being the queen of the entire DNA and RNA family and is treatment for all poxviridae diseases due to incorporation.
